(19) United States
(12) Patent Application Publication
(10) Pub. No.: US 2024/0018601 A1
(43) Pub. Date: Jan. 18, 2024

(54) NOVEL BIOMARKER FOR PREDICTING PROGNOSIS OF TREATMENT OF HER2-POSITIVE BREAST CANCER AND USE THEREOF

(71) Applicant: SAMSUNG LIFE PUBLIC WELFARE FOUNDATION, Seoul (KR)

(72) Inventors: Kyung Hee PARK, Seoul (KR); Yeon Hee PARK, Seoul (KR); Woong Yang PARK, Seoul (KR); Ji Yeon KIM, Seoul (KR)

(21) Appl. No.: 18/198,443

(22) Filed: May 17, 2023

(30) Foreign Application Priority Data
Jul. 14, 2022 (KR) 10-2022-0086734
Apr. 7, 2023 (KR) 10-2023-0046214

(51) Int. Cl. C12Q 1/6886; G01N 33/574

(52) U.S. Cl. CPC C12Q 1/6886; G01N 33/57415; C12Q 2600/106; C12Q 2600/156; C12Q 2600/158

(57) ABSTRACT

The present invention relates to a novel biomarker for predicting prognosis of treatment of HER2-positive breast cancer and the use thereof. The present invention presents the novel biomarker for predicting prognosis of treatment of HER2-positive breast cancer, and provides a composition and a kit for predicting prognosis of treatment of HER2-positive breast cancer, which can detect or measure the biomarker in HER2 early-positive breast cancer patients to whom neoadjuvant chemotherapy is applicable. Furthermore, according to the present invention, it is possible to select a patient-specific therapy by quickly and accurately determining the prognosis of drug treatment of the patient.

### BACKGROUND

### 1. Technical Field

The present invention relates to a novel biomarker for predicting prognosis of treatment of HER2-positive breast cancer and the use thereof.

### 2. Related Art

Breast cancer may be divided into four immunohistochemistry (IHC) types, including HR+HER2−, HR+HER2+, HR−HER2+, and triple-negative breast cancer (TNBC), based on the protein expressions of hormone receptor (HR) and human epidermal growth factor receptor 2 (HER2), and may be divided into five molecular types, including Luminal A, Luminal B, Her2-enriched, Basal-like, and Normal-like, according to PAM50 subtyping based on RNA expression levels. These types exhibit different clinical characteristics and responses to chemotherapy. HER2-positive breast cancer with HER2 amplification or overexpression accounts for 15 to 25% of all breast cancers, and has a high recurrence rate and poor prognosis. In HER2-positive breast cancer, HER2 is used as a biomarker or therapeutic target to predict breast cancer prognosis. Trastuzumab, a HER2-targeted therapeutic agent approved by the FDA, is a humanized monoclonal antibody against HER2, and is known to improve the disease-free survival rate and overall survival rate of HER2-positive breast cancer patients. However, only some patients respond to trastuzumab monotherapy and many patients are resistant to continuous treatment with trastuzumab, and to overcome this limitation of trastuzumab, various treatment strategies using trastuzumab in combination with other HER2-targeted therapeutic agents such as lapatinib and pertuzumab, cytotoxic anti-cancer agents, or immunotherapeutic anticancer agents have been attempted.

Anticancer therapies against breast cancer are broadly divided into radical chemotherapy (neoadjuvant chemotherapy or adjuvant chemotherapy) and palliative chemotherapy. Thereamong, neoadjuvant chemotherapy is a preoperative therapy which is used to reduce the tumor size to a size that can be operated or to lower the disease stage when the tumor cannot be removed or the surgical range is excessively large, and it may provide a good prognosis. In the case of HER2-positive early breast cancer, TCHP therapy using docetaxel, carboplatin, trastuzumab, and pertuzumab in combination increases the pathologic complete response rate to 60% or more. The TCHP therapy can control toxicity, but has the disadvantage of exhibiting adverse effects with a high risk of grade 3 or 4, including neutropenic fever, neurotoxicity, nephrotoxicity, emesis, and diarrhea. Therefore, since each patient has a different response to treatment, it is necessary to evaluate the association between molecular types of breast cancer and clinical results in order to accurately predict prognosis. In particular, it is necessarily required to conduct studies on molecular factors that affect the prognosis of each type of breast cancer.

### PRIOR ART DOCUMENTS

### Patent Documents

Korean Patent No. 10-1960431

Korean Patent No. 10-2338510

### SUMMARY

An object of the present invention is to provide a composition and a kit for predicting prognosis of treatment of HER2-positive breast cancer.

Another object of the present invention is to provide a method of providing information for predicting prognosis of treatment of HER2-positive breast cancer.

### Composition for Predicting Prognosis of Treatment of HER2-Positive Breast Cancer

One aspect of the present invention provides a composition for predicting prognosis of treatment of HER2-positive breast cancer, the composition containing an agent for detecting RAD21 gene.

“HER2-positive breast cancer” used in the present invention is a type of breast cancer in which HER2 is found more than in general cancer cells, and is characterized by a faster and more aggressive progression than other breast cancers. In the case of HER2-positive early breast cancer, the tumor size is 2 cm or less, there is no metastasis in the axillary lymph node, and it is known that the use of a HER2-targeted therapeutic agent provides good prognosis and a high cure rate. If the tumor size exceeds 2 cm or if there is lymph node metastasis, treatment with a targeted therapeutic agent is performed before surgery, and at this time, complete eradication of the tumor is called pathologic complete response (pCR).

As used herein, the term “treatment” refers to a therapy commonly used to inhibit the growth, proliferation or metastasis of breast cancer, and includes all cases in which a targeted therapeutic agent, a cytotoxic anticancer agent, or an immunotherapeutic anticancer agent is used alone or in combination. Common anticancer therapies include neoadjuvant chemotherapy intended to reduce the tumor size by administering anticancer drugs before surgery, adjuvant chemotherapy that administers anticancer drugs after surgery for the purpose of preventing recurrence of cancer due to a high likelihood that microscopic residual cancer will remain after radical resection (complete resection), and palliative chemotherapy intended to improve the patient's quality of life by slowing or alleviating disease progression and ultimately prolong the patient survival.

According to one embodiment of the present invention, the treatment of HER2-positive breast cancer may be for HER2-positive early breast cancer patients to whom neoadjuvant chemotherapy is applicable.

According to one embodiment of the present invention, the neoadjuvant chemotherapy may be performed using a HER2-targeted therapeutic agent alone or in combination with an anticancer agent such as a cytotoxic anticancer agent or an immunotherapeutic anticancer agent.

Examples of the HER2-targeted anticancer agent include, but are not limited to, dacomitinib, margetuximab, neratinib, pertuzumab, trastuzumab, trastuzumab emtansine, tucatinib, lapatinib, and the like.

Examples of the cytotoxic anticancer agent include, but are not limited to, cyclophosphamide, docetaxel, paclitaxel, nanoparticle albumin-bound paclitaxel (nab-paclitaxel), doxorubicin, and the like.

Examples of the immunotherapeutic anticancer agent include, but are not limited to, atezolizumab, ipilimumab, nivolumab, pembrolizumab, and the like.

More specifically, the neoadjuvant chemotherapy may be a TAHP therapy comprising administering: docetaxel, paclitaxel, or nanoparticle albumin-bound paclitaxel; trastuzumab; pertuzumab; and atezolizumab, nivolumab or pembrolizumab.

As used in the present invention, the term “prognosis” refers to determining whether recurrence, metastasis, drug response, drug resistance, etc. will occur before/after treatment in an individual who has not been diagnosed or has been diagnosed. In the present invention, the term “prognosis” means predicting whether the response to treatment will be good, by determining the presence or absence of the RAD21 gene as a biomarker, more specifically, mutation in the RAD21 gene, and/or the RNA expression level of the RAD21 gene before drug treatment in HER2-positive breast cancer patients, more specifically, HER2-positive early breast cancer patients to whom neoadjuvant chemotherapy is applicable, and the prognosis may be predicted using pathologic complete remission (pCR). As used herein, the term “biomarker” generally refers to any substance that is detectable in a biological sample, and examples thereof include all organic biomolecules such as polypeptides, proteins, nucleic acids, genes, lipids, glycolipids, glycoproteins, and sugars, which can indicate biological changes.

The composition for predicting prognosis of treatment of HER2-positive breast cancer according to the present invention requires an agent for detecting the RAD21 gene, which is a biomarker.

According to one embodiment of the present invention, the agent may be one for detecting a DNA mutation of the RAD21 gene, the RNA expression level of the RAD21 gene, or a combination thereof.

According to one embodiment of the present invention, the DNA mutation of the RAD21 gene may be at least one selected from the group consisting of i) a single nucleotide mutation; ii) deletion, substitution, insertion, or combination thereof, of 1 to 50 nucleotides, and iii) copy number variation in the nucleic acid sequence or nucleotide sequence of the RAD21 gene.

As used herein, the term “mutation or variation” refers to alteration of one or more bases, nucleotides, polynucleotides or nucleic acids in a genome. The mutation may include substitution, insertion, or deletion of one or more bases, nucleotides, polynucleotides, or nucleic acids. As used herein, the term “substitution” refers to an alteration in which one or more bases, nucleotides, polynucleotides or nucleic acids are replaced with other bases, nucleotides, polynucleotides or nucleic acids. The term “insertion” refers to an alternation in which one or more other bases, nucleotides, polynucleotides or nucleic acids are added. The term “deletion” refers to an alteration in which one or more bases, nucleotides, polynucleotides or nucleic acids are removed.

As used herein, “single nucleotide variation (SNV)” refers to a sequence alteration or mutation showing a difference of a single base or nucleotide in a genome, and may be used interchangeably with single nucleotide polymorphism, which means that one specific base is changed to another base at the same location in the genomes of several people and expressed as another trait. The expression “deletion or insertion of 1 to 50 nucleotides” refers to a sequence alteration or mutation showing a difference of 1 to 50 or more contiguous or non-contiguous bases, nucleotides, polynucleotides or nucleic acids in a genome. Such nucleotide variation may affect even one amino acid consisting of three bases, and base differences may contribute to differences between individuals, including susceptibility to specific diseases, disease expression patterns, and responsiveness to therapeutic agents.

As used herein, the term “copy number variation (CNV)” refers to a DNA segment with a length of 1 kb or more, which shows a difference in the number of repeated sequences when compared to a reference sequence. Differences in copy number may contribute to differences between individuals, including susceptibility to specific diseases, disease expression patterns, and responsiveness to therapeutic agents.

The presence or absence of such DNA mutations may be detected through sequencing or amplification reaction.

In one Example of the present invention, as a result of performing DNA and RNA sequencing using tumor tissues obtained before drug treatment of HER2-positive early breast cancer patients who received TAHP therapy, it was confirmed that the pCR rate was significantly higher in cases (78%) with wild-type RAD21 gene than in cases (24%) having a mutation in the RAD21 gene, more specifically, a copy number variation (RAD21_amp) in which the copy number increased by 6 or more. Based on this result, the present inventors formed predictive models having high performance of predicting prognosis of treatment of HER2-positive early breast cancer (RAD21_exprs+RAD21_amp, LOOCV.AUC=0.690; RAD21_amp, LOOCV.AUC=0.733; RAD21_exprs, LOOCV.AUC=0.749) only based on the DNA mutation and/or RNA expression level of the RAD21 gene and having excellent accuracy.

According to one embodiment of the present invention, the agent for detecting a DNA mutation in the RAD21 gene may be at least one selected from the group consisting of sense and antisense primers, probes, and antisense nucleotides, which bind complementarily to DNA of the RAD21 gene.

In addition, according to one embodiment of the present invention, the agent for detecting or measuring the RNA expression level of the RAD21 gene may be a primer or probe that binds complementarily to RNA of the RAD21 gene.

The composition for predicting prognosis of treatment of HER2-positive breast cancer according to the present invention, which contains the agent for detecting the RAD21 gene, may utilize other biomarkers in addition to RAD21 to improve prediction accuracy.

According to one embodiment of the present invention, the composition may further contain an agent for measuring the expression level of the HER2 gene or protein.

In one Example of the present invention, as a result of measuring the HER2 protein expression level using tumor tissue obtained before drug treatment of HER2-positive early breast cancer patients who received TAHP therapy, it was confirmed that the pCR rate was significantly higher in cases of HER2 3+ (71%) than in cases of HER2 2+ (13%). Based on this result, the present inventors formed a predictive model having high performance of predicting prognosis of treatment of HER2-positive early breast cancer based on the expression of HER2 together with the presence of DNA mutation in the RAD21 gene (LOOCV.AUC=0.807) and having excellent accuracy.

According to another embodiment of the present invention, the composition may further contain an agent for measuring the expression level of the PDL1 gene or protein.

The PDL1 (programmed death-ligand 1) is a protein on the surface of cancer cells or in hematopoietic cells, and PDL1 on the surface of cancer cells binds to PD-1 (programmed cell death protein 1) on the surface of T cells to enable cancer cells to escape from T cell attack.

In one Example of the present invention, as a result of measuring the PDL1 protein expression level using tumor tissues obtained before drug treatment of HER2-positive early breast cancer patients who received TAHP therapy, it was confirmed that the pCR rate was significantly higher in PDL1-positive cases (100%) than in PDL1-negative cases (55%). Based on this result, the present inventors formed predictive models having further improved performance of predicting prognosis of treatment of HER2-positive early breast cancer based on the expression of HER2 and/or PDL1 together with the DNA mutation and/or RNA expression level of the RAD21 gene (RAD21_amp+PDL1, LOOCV.AUC=0.757; RAD21_amp+HER2+PDL1+RAD21_exprs, LOOCV.AUC=0.788; RAD21_amp+HER2, LOOCV.AUC=0.807; RAD21_amp+HER2+PDL1, LOOCV.AUC=0.839) and having excellent accuracy.

In order to measure the expression level of HER2 or PDL1, an agent capable of measuring the expression level of the corresponding gene or protein is required.

According to one embodiment of the present invention, the agent for measuring the expression level of the HER2 or PDL1 gene or protein may be at least one selected from the group consisting of sense and antisense primers, probes, antisense nucleotides, antibodies, oligopeptides, ligands, peptide nucleic acids (PNAs), and aptamers, which bind complementarily or specifically to the HER2 or PDL1 gene or protein.

According to another embodiment of the present invention, the composition may further contain an agent for measuring the RNA expression level of at least one gene selected from the group consisting of ANKRD50, COX6C, DERL1, FLNB, GPRC5A, RNF139, SAMD8, SERPINE1, SQLE, and TTC39A genes.

The above 10 genes are selected genes confirmed to show a statistically significant difference in their level of expression in the non-pCR group versus the pCR group among HER2-positive breast cancer patients in one Example of the present invention.

In one Example of the present invention, as a result of measuring the RNA expression levels of the 10 genes using tumor tissue obtained before drug treatment of HER2-positive early breast cancer patients who received TAHP therapy, the present inventors formed predictive models having further improved performance of predicting prognosis of treatment of HER2-positive early breast cancer based on the expression of HER2 and/or PDL1, and/or the RNA expression levels of the 10 genes, together with the DNA mutation and/or RNA expression level of the RAD21 gene (including RAD21_amp and/or RAD21_exprs, LOOCV.AUC=0.734 to 0.918), and having excellent accuracy.

In order to measure the expression level of these 10 genes, agents capable of measuring the RNA expression levels of the corresponding genes are required.

According to one embodiment of the present invention, the agents for measuring the RNA expression levels of the 10 genes may be primers or probes that complementarily bind to the RNAs of the genes, respectively.

As used herein, the term “primer” refers to a nucleic acid sequence having a short free 3′-end hydroxyl group, which is a single-stranded oligonucleotide that is capable of forming a base pair with a complementary template and functions as a start point for template strand replication. The primer may initiate DNA synthesis in the presence of a reagent for polymerization (e.g., DNA polymerase or reverse transcriptase) and four different nucleoside triphosphates in suitable buffer at a suitable temperature. The primer pair consists of sense and antisense oligonucleotide primers having a sequence of 7 to 50 nucleotides, and may have a sequence of 15 to 30 nucleotides within a range that does not alter the basic properties of the primer that serves as an initiation point for DNA synthesis.

As used herein, the term “probe” refers to a linear oligomer of natural or modified monomers or linkages, including deoxyribonucleotides and ribonucleotides, which is capable of specifically hybridizing with a target nucleotide sequence, whether occurring naturally or produced synthetically.

Such primers, probes and antisense nucleotides may, if necessary, contain a label detectable directly or indirectly by spectroscopic, photochemical, biochemical, immunochemical, or chemical means. The detectable label is a label substance capable of generating a detectable signal, and examples thereof include label substances capable of generating a detectable signal, including fluorophores such as Cy3 or Cy5. The detectable label can identify nucleic acid hybridization results.

As used herein, the term “antibody” refers to a specific protein molecule that is directed against an antigenic site. In the present invention, the term “antibody” refers to an antibody that specifically binds to HER2 or PDL1 protein, and includes all of monoclonal antibodies, polyclonal antibodies, and recombinant antibodies. Here, the expression “binds specifically” means that the binding affinity to a target substance is superior to the binding affinity to other substances to the extent that the presence or absence of the target substance can be detected by binding. In addition, the antibodies include not only complete forms having two full-length light chains and two full-length heavy chains, as well as functional fragments of antibody molecules. The expression “functional fragments of antibody molecules” refers to fragments retaining at least an antigen-binding function, and examples of the functional fragments of antibody molecules include Fab, F(ab′), F(ab′)2, Fv, and the like.

The antibodies may be easily produced through techniques known in the art. For example, monoclonal antibodies may be produced using the hybridoma method well known in the art (see Kohler and Milstein (1976) European Journal of Immunology 6:511-519), or a phage antibody library technique (Clackson et al, Nature, 352:624-628, 1991; Marks et al, J. Mol. Biol., 222:58, 1-597, 1991). Polyclonal antibodies may be produced by a method of injecting a target protein antigen into an animal and collecting blood from the animal to obtain serum containing the antibody. Such polyclonal antibodies may be produced from animals such as goats, rabbits, sheep, monkeys, horses, pigs, cows, or dogs.

The antibody produced by the above-described method may be separated and purified using methods such as gel electrophoresis, dialysis, salt precipitation, ion exchange chromatography, or affinity chromatography.

### Kit for Predicting Prognosis of Treatment of HER2-Positive Breast Cancer

Another aspect of the present invention provides a kit for predicting prognosis of treatment of HER2-positive breast cancer, the kit comprising the composition.

As used herein, the expression “kit for predicting prognosis of treatment of HER2-positive breast cancer” refers to a substance capable of predicting prognosis through a biological sample isolated from a test subject or a HER2-positive breast cancer patient, more specifically, a HER2-positive early breast cancer patient to whom neoadjuvant chemotherapy is applicable. Through the kit, it is possible to diagnose the prognosis of a test subject after treatment quickly, accurately and conveniently. In the present invention, the kit may comprise an agent for detecting or measuring the DNA mutation and/or RNA expression level of the RAD21 gene, or may further comprise, in addition to the above agent, an agent for measuring the expression level of the HER2 or PDL1 gene or protein, and/or agents for measuring the RNA expression levels of the above-described 10 genes.

Examples of the kit include, without limitation, conventional diagnostic kits based on gene expression, gene mutation (e.g., copy number variation), RNA (e.g., mRNA) expression, and protein quantitative analysis.

According to one embodiment of the present invention, the kit may be at least one selected from the group consisting of a polymerase chain reaction (PCR) kit, a reverse transcription PCR (RT-PCR) kit, a DNA or DNA chip kit, a next generation sequencing (NGS) kit, a protein chip kit, and a protein array kit.

For example, when the kit of the present invention is applied to a PCR amplification process, the kit may optionally comprise reagents required for PCR amplification, such as buffer, DNA polymerase, DNA polymerase cofactor, and dNTPs, and when the kit of the present invention is applied to immunoassay, the kit may optionally comprise a substrate for secondary antibody and a label. In addition, the kit according to the present invention may be made of a plurality of separate packagings or compartments including the above reagent components, and the kit of the present invention may be a diagnostic kit comprising essential elements necessary for performing DNA chip assay. The DNA chip kit may comprise a substrate to which a cDNA corresponding to a gene or a fragment thereof is attached as a probe, and a reagent, an agent, an enzyme and the like for constructing a fluorescence-labeled probe. In addition, the substrate may comprise a cDNA corresponding to a control gene or a fragment thereof.

### Method of Providing Information for Predicting Prognosis of Treatment of HER2-Positive Breast Cancer

Another aspect of the present invention provides a method of providing information for predicting prognosis of treatment of HER2-positive breast cancer, the method comprising steps of: a) detecting RAD21 gene in a biological sample isolated from a subject; and b) comparing the DNA mutation level, RNA expression level, or combination thereof, of the detected RAD21 gene with that in a control group.

Steps a) and b) will be described below in detail, and the description of contents overlapping with those described above will be omitted to avoid excessive complexity.

Step a) is a process of collecting a biological sample from an individual or subject to be tested for prognosis after treatment of HER2-positive breast cancer and measuring the DNA mutation level and/or RNA expression level of the RAD21 gene in the biological sample.

According to one embodiment of the present invention, the subject in step a) may be a HER2-positive early breast cancer patient to whom neoadjuvant chemotherapy is applicable.

More specifically, the neoadjuvant chemotherapy may comprise administration of: docetaxel, paclitaxel or nanoparticle albumin-bound paclitaxel (nab-paclitaxel); trastuzumab; pertuzumab; and atezolizumab, nivolumab or pembrolizumab.

According to one embodiment of the present invention, the biological sample in step a) may be at least one selected from the group consisting of blood, plasma, serum, lymphatic fluid, saliva, urine, and tissue.

The biological sample is preferably obtained before receiving drug treatment for HER2-positive breast cancer. In order to accurately measure the genetic mutation of cancer cells and the expression level of molecules (e.g., nucleic acids, proteins) from the biological sample, cancer tissue is preferably used.

Step b) is a process of predicting prognosis after treatment of HER2-positive breast cancer in a subject or individual based on the specific mutation and/or RNA expression level of the RAD21 gene, measured in the biological sample.

According to one embodiment of the present invention, the DNA mutation in the RAD21 gene may be at least one selected from the group consisting of: a single nucleotide mutation; deletion, substitution, insertion, or combination thereof, of 1 to 50 nucleotides; and copy number variation in the RAD21 gene sequence.

In addition, the method of providing information for predicting prognosis of treatment of HER2-positive breast cancer according to the present invention may further comprise a step of measuring the expression levels of HER2, PDL1, and/or at least one gene or protein selected from the group consisting of ANKRD50, COX6C, DERL1, FLNB, GPRC5A, RNF139, SAMD8, SERPINE1, SQLE, and TTC39A, in addition to the expression level of the RAD21 gene.

More specifically, the method may further comprise, after step a) or b), steps of: a-1) measuring the expression level of the HER2 gene or protein in the biological sample; and b-1) comparing the measured expression level of the HER2 gene or protein with that in a control group.

In addition, the method may further comprise, after step a) or b), steps of: a-2) of measuring the expression level of the PDL1 gene or protein in the biological sample; and b-2) comparing the measured expression level of the PDL1 gene or protein with that in a control group.

In addition, the method may further comprise, after step a) or b), steps of: a-3) measuring the RNA expression level of at least one gene selected from the group consisting of ANKRD50, COX6C, DERL1, FLNB, GPRC5A, RNF139, SAMD8, SERPINE1, SQLE, and TTC39A genes, in the biological sample; and b-3) comparing the measured RNA expression level of the gene with that in the control group.

According to one embodiment of the present invention, the DNA mutation level of the RAD21 gene may be analyzed by fluorescence in situ hybridization, chromatin immunoprecipitation, next-generation sequencing (NGS), or the like, without being limited thereto.

More specifically, DNA mutation of the RAD21 gene, particularly copy number variation, may be measured by NGS, for example, whole genome sequencing, whole exome sequencing, target gene panel sequencing, or the like, without being limited thereto.

The NGS has multiplexing ability to simultaneously perform hundreds of thousands of reactions, and enables sequencing even with a small amount of sample. NGS technologies have slightly different specific application techniques depending on commercialized technologies, but generally use new sequencing methods having mechanisms of action different from clonal amplification, massively parallel sequencing, and Sanger methods. Examples of the commercialized technologies include the 454 GS improved FLX model sequencer released by Roche in 2007, the Genome Analyzer HiSeq released by Illumina in 2006, and SOLiD released by Applied Biosystems in 2007. In common, these three platforms abandoned complex library construction and cloning processes and adopted clonal amplification technology. In addition, these platforms adopted massively parallel sequencing technology capable of high-throughput processing at once, and eliminated the complicated electrophoresis process by determining sequences by sequencing-by-synthesis through cyclic sequencing. In addition, they use an algorithm that arranges short reads, obtained using the shotgun method, with a computer, finds overlapping parts, and completes the whole. NGS may be clinically used for genetic panel testing, exome sequencing, whole genome sequencing, single nucleotide polymorphism detection, blood-based tumor diagnostics, noninvasive prenatal testing, human leukocyte antigen testing, immunoglobulin rearrangement testing, RNA sequencing, DNA methylation testing, chromatin immunoprecipitation (ChIP) sequencing, single cell sequencing, etc.

According to one embodiment of the present invention, the expression level of the gene (e.g., DNA, RNA) may be measured by methods such as polymerase chain reaction (PCR), reverse transcription polymerase chain reaction (RT-PCR), competitive RT-PCR, real-time PCR, real-time RT-PCR, nuclease protection assay, in situ hybridization, DNA or RNA microarray, Northern blotting, Southern blotting, next-generation sequencing (NGS), etc., without being limited thereto.

In addition, according to one embodiment of the present invention, the expression level of the protein encoded by the gene may be measured determined by methods such as enzyme-linked immunosorbent assay, Western blotting, radioimmunoassay, radioimmunodiffusion, immunoprecipitation, flow cytometry, immunohistochemistry, immunofluorescence, protein microarray, etc., without being limited thereto.

The DNA mutation level and/or RNA expression level of the RAD21 gene, the expression level of the HER2 and/or PDL1 gene or protein, and/or the RNA expression levels of the 10 genes, detected or measured as described above, may be analyzed comparatively with the mutation level and/or the expression level of the gene in a control group, more specifically, a healthy person sample, thereby predicting prognosis of treatment of the HER2-positive breast cancer patient.

According to one embodiment of the present invention, in the step of comparing with that in the control group, it may be determined that, when one or more of the following i) and ii) and one or more of the following iii) to v) are satisfied in the subject compared to the control group, the prognosis of treatment of the HER2 positive breast cancer patient is good:
i) a low DNA mutation level of the RAD21 gene;
ii) a low RNA expression level of RAD21 gene;
iii) a high expression level of the HER2 gene or protein;
iv) a high expression level of the PDL1 gene or protein; and
v) a low RNA expression level of at least one selected from the group consisting of ANKRD50, COX6C, DERL1, FLNB, GPRC5A, RNF139, SAMD8, SERPINE1, SQLE and TTC39A genes.

Here, “low DNA mutation level of the RAD21 gene” means that the number of copy number variations among mutations is less than 6 or 0, and “low RNA expression level of the RAD21 gene” means that the expression level of mRNA transcribed from the DNA encoding the gene is low. In addition, “high expression level of the HER2 gene or protein” means that the expression level of the protein or the gene encoding the same is high (specifically, 3+ or higher), which may be due to DNA mutation (e.g., copy number variation) of the HER2 gene, and “high expression level of the PDL1 gene or protein” means that the protein or the gene encoding the same is expressed (positive). In addition, “low RNA expression level of at least one of the 10 genes” means that the expression level of mRNA transcribed from the DNA encoding each of the genes is low.

More specifically, when the subject has a low DNA mutation level and/or RNA expression level of the RAD21 gene while having a high expression level of the HER2 gene or protein, and/or a high expression level of the PDL1 gene or protein, and/or a low RNA expression level of at least one selected from the group consisting of ANKRD50, COX6C, DERL1, FLNB, GPRC5A, RNF139, SAMD8, SERPINE1, SQLE and TTC39A genes, compared to the control group, the prognosis of treatment of HER2-positive early breast cancer in the subject may be predicted to be good, and performance and accuracy of the prediction may be improved.

The present invention presents the novel biomarker for predicting prognosis of treatment of HER2-positive breast cancer, and provides a composition and a kit for predicting prognosis of treatment of HER2-positive breast cancer, which can detect or measure the biomarker in HER2 early-positive breast cancer patients to whom neoadjuvant chemotherapy is applicable. Furthermore, according to the present invention, it is possible to select a patient-specific therapy by quickly and accurately determining the prognosis of drug treatment of the patient.

### BRIEF DESCRIPTION OF THE DRAWINGS

FIG. 1 shows the results of Fisher's exact tests for eight candidate biomarkers (DNA markers: RAD21, MYC, MYCN and ERBB2; RNA marker: Luminal; and protein markers: HR, HER2 and PDL1) for predicting prognosis of treatment of HER2-positive breast cancer according to one Example of the present invention.

FIG. 2 is a graph showing the LOOCV.AUC (Leave-One-Out Cross Validation Area Under the Curve) and prediction error of a generalized linear model (GLM) for a combination of the eight candidate biomarkers according to one Example of the present invention.

FIG. 3 shows ROC curves for a single biomarker (RAD21_amp) or biomarker combinations (HER2+RAD21_amp and HER2+PDL1+RAD21_amp) for predicting prognosis of treatment of HER2-positive breast cancer according to one Example of the present invention.

FIG. 4 shows the results of Fisher's exact test for the association between copy number variation (RAD21_amp) and RNA expression level (RAD21_exprs) of the RAD21 gene according to one Example of the present invention.

FIG. 5 is a graph showing the LOOCV.AUC (Leave-One-Out Cross Validation Area Under the Curve) and prediction error of a generalized linear model (GLM) versus a combination of the DNA marker (RAD21_amp), protein markers (HER2 and PDL1) and RNA markers (ANKRD50_exprs, COX6C_exprs, DERL1_exprs, FLNB_exprs, GPRC5A_exprs, RAD21_exprs, RNF139_exprs, SAMD8_exprs, SERPINE1_exprs, SQLE_exprs and TTC39A_exprs) according to one Example of the present invention.

FIG. 6 shows ROC curves for a single RNA marker (RAD21_exprs) or combinations of DNA marker, protein marker and RNA marker (SERPINE1_exprs+HER2+PDL1+RAD21_amp, and TTC39A_exprs+SQLE_exprs+SERPINE1_exprs+DERL1_exprs+ANKRD50_exprs+HER2+PDL1+RAD21_amp) for predicting prognosis of treatment of HER2-positive breast cancer according to one Example of the present invention.

### DETAILED DESCRIPTION

Hereinafter, the present invention will be described in more detail with reference to examples. However, these examples are merely provided to facilitate understanding of the present invention, and the scope of the present invention is not limited by these examples.

### EXAMPLE 1

### Selection of Biomarker Candidates for Predicting Prognosis of Treatment of HER2-Positive Breast Cancer

### 1-1. Preparation of Patient Samples

67 patients with HER2-positive early breast cancer who underwent neoadjuvant chemotherapy during a period from May 2019 to May 2020 were recruited, and 65 of them underwent curative surgery. Data collection ended at the end of October 2020, when the last patient underwent curative surgery. The neoadjuvant chemotherapy was TAHP therapy, in which docetaxel, trastuzumab, pertuzumab, and atezolizumab were administered for 6 cycles.

Of the 67 patients, 32 were HR-positive, and the median age was 52 years (ranging from 33 to 74 years). In patient biopsies before TAHP therapy, 13 patients (19.7%) were PDL1-positive by SP142 Ab. Overall, the pathologic complete remission (pCR) rate was 61.2% (41/67), and the non-pathologic complete remission (non-pCR) rate was 35.8% (24/67). One patient was unable to undergo surgery due to a relapse during TAHP therapy. Neutropenia was found in 13 patients (19.4%), but neutropenic fever was found in 5 patients (7.5%). In addition, rash, the most common immune-related toxicity, appeared in 43 patients (64.2%, including 1 patient with Grade 3), encephalitis (Grade 3) in 1 patient, immune-related hepatitis in 2 patients (Grade 2 and Grade 3), pneumonia in 6 patients (not Grade 3 or Grade 4), and thyroid dysfunction in 6 patients, indicating a high complete remission rate and moderate toxicity.

Tumor tissues were collected from all of the patients before TAHP therapy (T1; n=63, 40 pCR and 23 non-pCR), and from residual tumor tissues (T3; n=15) of patients who did not show pathologic complete remission (non-pCR) after surgery. Candidate biomarkers were selected by performing DNA and RNA sequencing and immunohistochemistry (IHC) using the collected tumor tissues.

### 1-2. Selection of DNA Markers

DNA was extracted from tumor tissue and DNA markers were screened by NGS. First, a whole-genome shotgun library was prepared from genomic DNA extracted using the QIAamp DNA Mini Kit (Qiagen). After probe hybridization, deep sequencing was performed with the Illumina® HiSeq 4000 System using the FoundationOne® CDx (F1CDx) panel. After sequencing, DNA mutations such as SNV, Indel (insertion+deletion), CNV, and fusion were analyzed using FoundationOne CDx™ software. Cells with low quality were excluded, and only mutations with a mutant allele frequency (MAF) of 5% or more (MAF of 1% or more in the case of a hotspot mutation) were reported. The association between genomic features and pCR achievement was analyzed by Fisher's exact test for the enrichment test by mutations. Among mutations, copy number amplification refers to a copy number of 6 or more compared to a process-matched normal control (see PMA P170019: FDA Summary of Safety and Effectiveness Data). A p-value<0.05 was considered statistically significant.

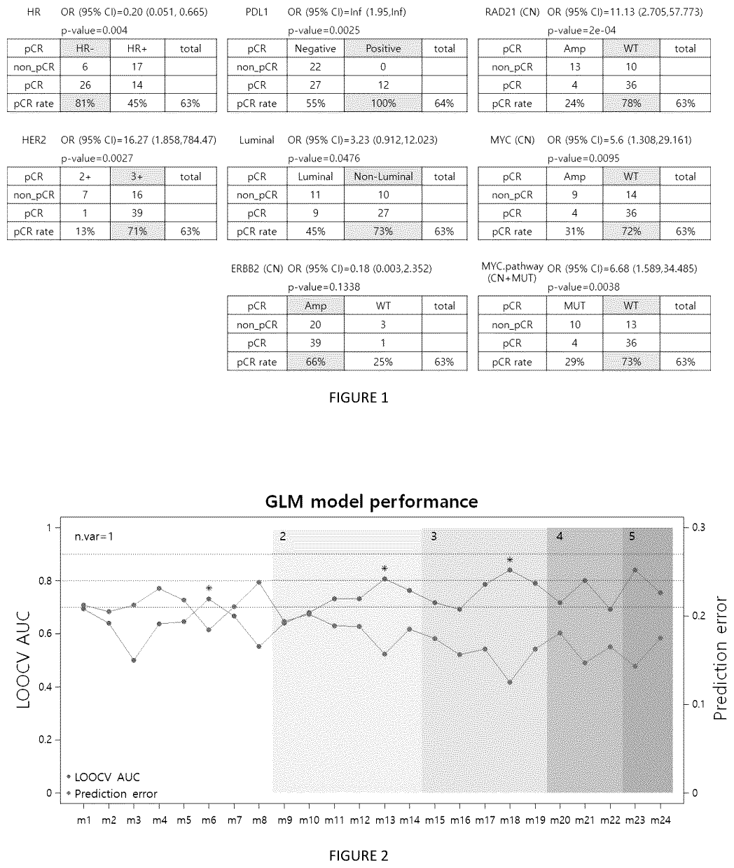

As a result, referring to FIG. 1, among 32 DNA markers, RAD21 copy number amplification (n=17, p=0.0002), MYC copy number amplification (n=13, p=0.0095) and MYC pathway mutations (MYC copy number amplification or MYCN mutation) (n=14, p=0.0095) were more frequently detected in the non-pCR group (n=23) than in the pCR group (n=40). In particular, wild type (WT) RAD21 without mutation showed a high pCR rate of 78% compared to the other markers. In addition, in the case of the ERBB2 gene encoding HER2, the ERBB2 copy number amplification (n=59, p=0.1338) did not significantly differ between the two groups, but co-amplification for ERBB2 and MYC was highly frequently detected in the non-pCR group (p=0.01687).

### 1-3. Selection of RNA Markers

RNA was extracted from tumor tissue and RNA markers were screened by NGS. First, total RNA was extracted from tumor tissue using a RNeasy Mini Kit (QIAGEN, USA) (for frozen tissue) or a Promega Relia Prep FFPE Total RNA Miniprep System kit (Promega, USA) (for FFPE tissue), and RNA integrity was measured using a 2100 Bioanalyzer (Agilent, USA). Whole transcriptome sequencing was performed to analyze the whole gene expression pattern. An RNA sequencing library was constructed using the TruSeq RNA Access Library Prep Kit (Illumina, Inc.) according to the manufacturer's instructions. In addition, paired-end sequencing was performed using the HiSeq 2500 Sequencing Platform (Illumina, Inc.) to convert the RNA library into sequencing reads and generate a FASTQ file. After removing poor-quality reads from the FASTQ file, the sequencing reads were aligned to the human reference genome (hg19) using STAR software (v2.5.2b), and RSEM software (v1.3) was used to measure the expression (read count and TPM) of each gene. PAM50 subtypes for research were predicted using genefu R package based on the gene expression data.

As a result, referring to FIG. 1, Luminal (patients belonging to Luminal A and Luminal B subtypes among PAM50 subtypes) of luminal tumors expressing HR estrogen receptor and progesterone receptor did not significantly differ between the pCR group and the non-pCR group, but non-Luminal (patients belonging to Her2-enriched, Basal-like and Normal-like subtypes among PAM50 subtypes) was more highly detected in the pCR group than in the non-pCR group.

### 1-4. Selection of Protein Markers

IHC was performed on tumor tissue to screen protein markers. Cell surface receptors ER, PR, HER2, and PDL1 proteins from each tissue were stained according to a conventional method and analyzed by a single pathologist. The case where the immunoreactivity of immune cells was 1% or more in the cancer area was determined to be PDL1-positive.

As a result, referring to FIG. 1, HR and HER2, which are conventional markers of HER2-positive breast cancer, were detected, and PDL1 was also detected.

### EXAMPLE 2

### Analysis I of Predictive Models for Candidate Biomarkers

For the candidate DNA markers RAD21, MYC, MYCN and ERBB2, the RNA marker Luminal, and the protein markers HR, HER2 and PDL1, selected in Example 1, predictive models were constructed by performing logistic regression of a generalized linear model (GLM) for a single marker or a combination of markers. The results are shown in Table 1 below and FIG. 2. In the evaluation of the performance of the markers, AUC (area under the curve) was used as an indicator, the LOOCV (leave-one-out cross validation) method was applied, and a model including the minimum number of markers while having LOOCV.AUC≥0.7, high accuracy and low prediction error was selected.

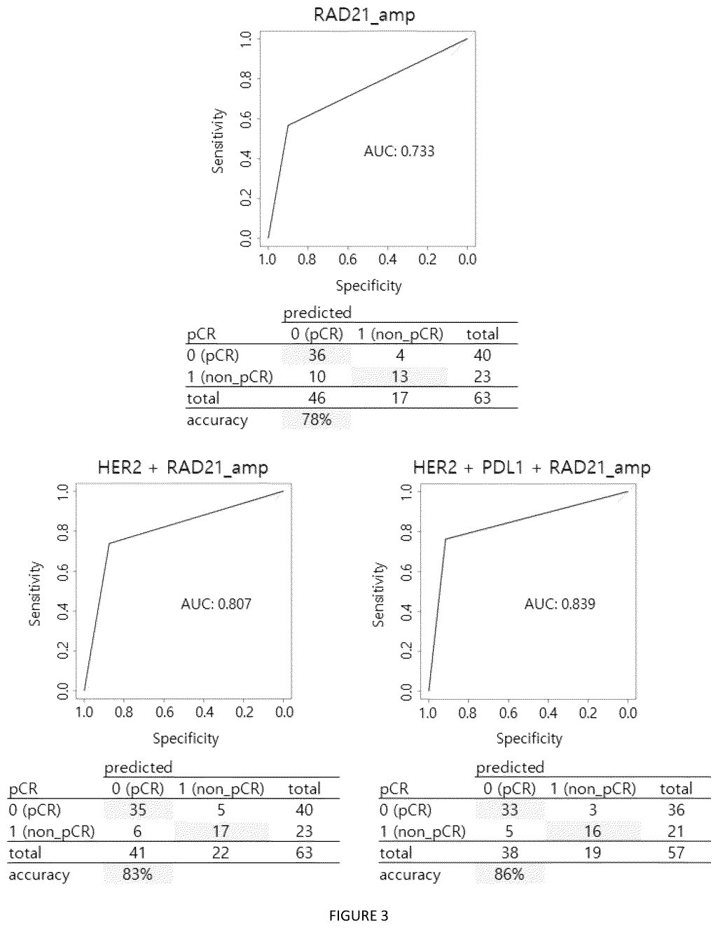

As a result, the model including RAD21_amp as a single marker exhibited excellent predictive performance compared to the conventional clinical marker HR or HER2 model, and the predictive performance of the model including, in addition to RAD21_amp, HER2 and additionally PDL1, was further improved (see FIG. 3).

TABLE 1

| | | | Prediction | | |
|---|---|---|---|---|---|
| | Model | AUC | error | LOOCV.AUC | Accuracy |
| m1 | HR | 0.695 | 0.212 | 0.695 | 0.683 |
| m2 | HER2 | 0.640 | 0.205 | 0.640 | 0.730 |
| m3 | PDL1 | 0.639 | 0.213 | 0.500 | 0.639 |
| m4 | Luminal | 0.637 | 0.231 | 0.637 | 0.667 |
| m5 | MYC | 0.646 | 0.218 | 0.646 | 0.714 |
| m6 | RAD21_amp | 0.733 | 0.185 | 0.733 | 0.778 |
| m7 | MYC.pathway | 0.667 | 0.210 | 0.667 | 0.730 |
| m8 | ERBB2 | 0.553 | 0.238 | 0.553 | 0.666 |
| m9 | HR + HER2 | 0.759 | 0.194 | 0.640 | 0.730 |
| m10 | PDL1 + Luminal | 0.745 | 0.202 | 0.679 | 0.719 |
| m11 | MYC + RAD21_amp | 0.746 | 0.189 | 0.733 | 0.778 |
| m12 | MYC.pathway + RAD21_amp | 0.748 | 0.188 | 0.733 | 0.778 |
| m13 | HER2 + RAD21_amp | 0.819 | 0.157 | 0.807 | 0.825 |
| m14 | ERBB2 + RAD21_amp | 0.769 | 0.185 | 0.764 | 0.794 |
| m15 | PDL1 + RAD21_amp | 0.820 | 0.159 | 0.757 | 0.803 |
| m16 | HER2 + PDL1 | 0.764 | 0.172 | 0.646 | 0.738 |
| m17 | HER2 + PDL1 + luminal | 0.803 | 0.174 | 0.716 | 0.754 |
| m18 | PDL1 + luminal + RAD21_amp | 0.870 | 0.156 | 0.693 | 0.737 |
| m19 | HR + HER2 + RAD21_amp | 0.870 | 0.163 | 0.785 | 0.810 |
| m20 | HER2 + PDL1 + RAD21_amp | 0.884 | 0.125 | 0.839 | 0.860 |
| m21 | ERBB2 + PDL1 + RAD21_amp | 0.844 | 0.163 | 0.790 | 0.820 |
| m22 | HR + HER2 + PDL1 + luminal | 0.825 | 0.181 | 0.716 | 0.754 |
| m23 | HR + HER2 + MYC + RAD21_amp | 0.891 | 0.147 | 0.802 | 0.810 |
| m24 | PDL1 + luminal + MYC + RAD21_amp | 0.882 | 0.165 | 0.693 | 0.737 |
| m25 | HR + HER2 + PDL1 + | 0.913 | 0.143 | 0.839 | 0.860 |
| | luminal + MYC + RAD21_amp | | | | |
| m26 | HR + ERBB2 + PDL1 + | 0.886 | 0.175 | 0.754 | 0.789 |
| | luminal + MYC + RAD21_amp | | | | |

These results suggest that RAD21 can be used as a biomarker to predict prognosis of drug treatment of HER2 early-positive breast cancer patients to whom neoadjuvant chemotherapy is applicable, and when there is no DNA mutation in the RAD21 gene, in particular, when there is no copy number amplification in the RAD21 gene or when the copy number of the RAD21 gene is less than 6, the prognosis of drug treatment can be predicted to be good, and when the expression levels of HER2 and/or PDL1 protein(s) are high, the prediction accuracy is excellent.

### EXAMPLE 3

### Selection of RNA Marker Candidates for Predicting Prognosis of Treatment of HER2-Positive Breast Cancer

New RNA markers were screened by NGS using the same patient samples and tumor tissues as in Example 1. RNA sequencing was performed in the same manner as in Examples 1-3.

DEG analysis was performed to screen genes whose expression level differs depending on whether pCR was present. After converting the gene expression level (TPM) into log 2, differentially expressed genes (DEGs) whose expression was statistically significantly higher in the non-pCR group than in the pCR group were selected. Using t test, genes, whose expression level was statistically significantly higher in the non-pCR group (p value<0.01) and did significantly differ between the groups (log 2 fold change>0.05) and which had a high average expression level (log 2TPM>3), were selected. A total of 16,726 genes were analyzed, 22 genes satisfying the above criteria were selected as candidate biomarkers, and the results are shown in Table 2 below.

TABLE 2

| | | pCR | non_pCR | | | |
|---|---|---|---|---|---|---|
| | | average | average | Expression | | |
| Gene | RNA marker name | expression | expression | difference | p value | REFSEQ |
| ANKRD50 | ANKRD50_exprs | 4.444 | 5.069 | 0.625 | 0.0032 | NM_020337 |
| ATAD2 | ATAD2_exprs | 4.671 | 5.336 | 0.665 | 0.0091 | NM_014109 |
| C8orf76 | C8orf76_exprs | 3.689 | 4.233 | 0.544 | 0.0082 | NM_032847 |
| COX6C | COX6C_exprs | 5.962 | 6.848 | 0.886 | 0.0014 | NM_004374 |
| DERL1 | DERL1_exprs | 5.558 | 6.226 | 0.668 | 0.0065 | NM_024295 |
| EIF3H | EIF3H_exprs | 6.872 | 7.655 | 0.782 | 0.0037 | NM_003756 |
| FLNB | FLNB_exprs | 6.337 | 6.877 | 0.540 | 0.0032 | NM_001457 |
| GPRC5A | GPRC5A_exprs | 3.956 | 5.078 | 1.121 | 0.0061 | NM_003979 |
| KITLG | KITLG_exprs | 3.677 | 4.324 | 0.647 | 0.0077 | NM_000899 |
| MRPL13 | MRPL13_exprs | 4.235 | 4.913 | 0.678 | 0.0030 | NM_014078 |
| OCLN | OCLN_exprs | 6.575 | 7.308 | 0.732 | 0.0066 | NM_002538 |
| RAD21 | RAD21_exprs | 7.110 | 7.927 | 0.818 | 0.0011 | NM_006265 |
| RNF139 | RNF139_exprs | 3.855 | 4.445 | 0.590 | 0.0005 | NM_007218 |
| RPL8 | RPL8_exprs | 9.934 | 10.466 | 0.532 | 0.0081 | NM_000973 |
| SAMD8 | SAMD8_exprs | 3.215 | 3.785 | 0.570 | 0.0077 | NM_144660 |
| SERPINE1 | SERPINE1_exprs | 3.992 | 4.627 | 0.635 | 0.0063 | NM_000602 |
| SLC39A4 | SLC39A4_exprs | 4.028 | 4.761 | 0.734 | 0.0088 | NM_017767 |
| SQLE | SQLE_exprs | 5.004 | 5.796 | 0.792 | 0.0060 | NM_003129 |
| TATDN1 | TATDN1_exprs | 4.654 | 5.189 | 0.535 | 0.0046 | NM_032026 |
| TRPS1 | TRPS1_exprs | 8.164 | 9.014 | 0.849 | 0.0092 | NM_014112 |
| TTC39A | TTC39A_exprs | 4.098 | 4.856 | 0.758 | 0.0065 | NM_001080494 |
| WASHC5 | KIAA0196_exprs | 5.891 | 6.457 | 0.566 | 0.0058 | NM_014846 |

Meanwhile, since the RAD21 gene was also included in the selected candidate RNA markers, the correlation between DNA mutation and RNA expression for the RAD21 gene was analyzed.

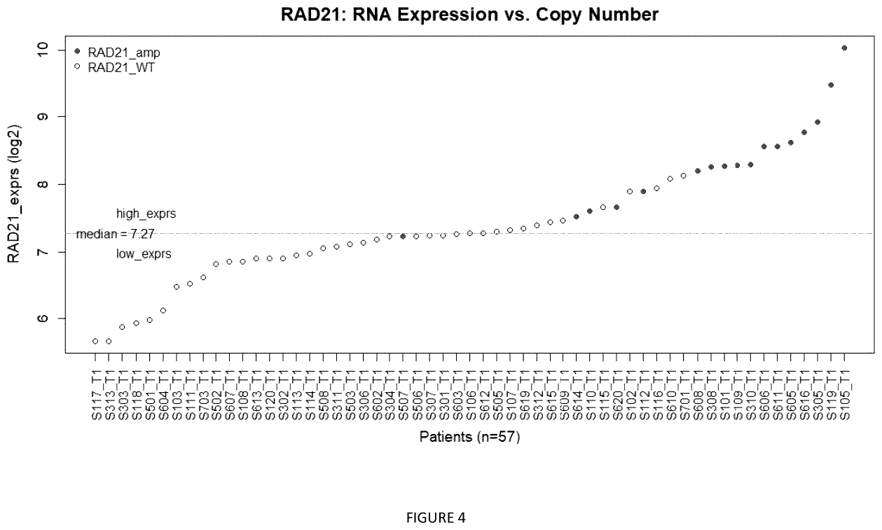

57 patients who obtained both DNA sequencing and RNA sequencing results were classified by the presence or absence of the copy number variation of the RAD21 gene and the RNA expression level (high expression/low expression) of the RAD21 gene based on the median value of RNA expression level, and the association between them was analyzed by Fisher's exact test (concordance rate=77%, p-value=6.843e-06). The results are shown in Table 3 below and FIG. 4.

TABLE 3

| | | RAD21 RNA expression level (RAD21_exprs) | | |
|---|---|---|---|---|
| | | High expression | Low expression | |
| | | (high_exprs) | (low_exprs) | Total |
| RAD21 | Copy number | 16 | 1 | 17 |
| DNA mutation | variation | | | |
| | (RAD21_amp) | | | |
| | Wild-type | 12 | 28 | 40 |
| | (RAD21_WT) | | | |
| | Total | 28 | 29 | 57 |

As a result, when the RAD21 gene had copy number variation, the RNA expression level thereof was also high, but the high RNA expression level of the RAD21 gene did not necessarily mean that the RAD21 gene had copy number variation. Therefore, it was confirmed that the association between the copy number variation of the RAD21 gene and the RNA expression level thereof was not statistically significant, and thus the DNA marker (RAD21_amp) and the RNA marker (RAD21_exprs) for RAD21 could be used as individual markers.

### EXAMPLE 4

### Analysis II of Predictive Models for Candidate Biomarkers

In order to improve the performance of the model for predicting the prognosis of treatment of HER2-positive breast cancer patients, new predictive models were constructed by adding the 22 RNA markers selected in Example 3 to the DNA marker RAD21 (RAD21_amp) and protein markers (HER2 and PDL1) selected in Example 2 and performing logistic regression of a generalized linear model (GLM). Among 63 patients with HER2+PDL1+RAD21_amp, 57 patients for whom both DNA sequencing and RNA sequencing results were obtained were analyzed. The presence or absence of mutations as DNA sequencing results and the gene expression levels as RNA sequencing results were input to the model, and optimal marker combinations were selected through forward/backward stepwise selection in logistic regression analysis. In the evaluation of the performance of the markers, AUC was used as an indicator, the LOOCV method was applied, and a model including the minimum number of markers while having LOOCV.AUC≥0.7, high accuracy and low prediction error was selected. In addition, among the candidate RNA markers, 11 genes whose performance was LOOCV.AUC>0.7 as individual markers or whose AUC was improved by 0.05 or more when individual markers were added to DNA markers were finally selected as RNA markers.

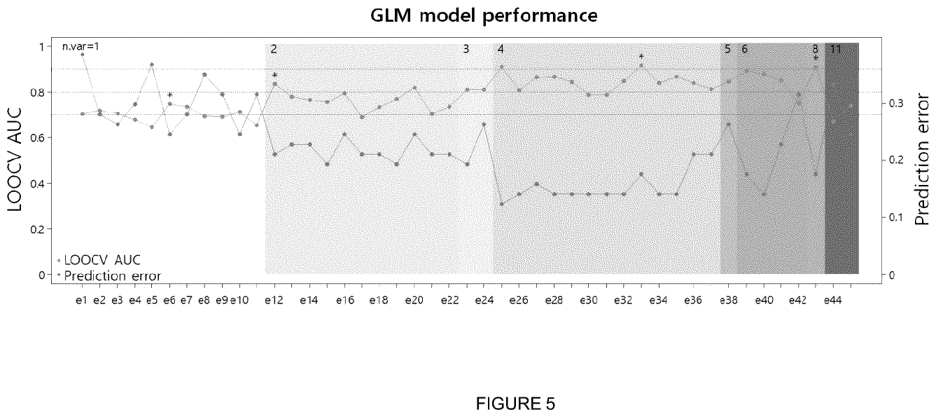

Predictive models for DNA marker RAD21 (RAD21_amp), protein markers HER2 and PDL1, and RNA markers ANKRD50, COX6C, DERL1, FLNB, GPRC5A, RAD21 (RAD21_exprs), RNF139, SAMD8, SERPINE1, SQLE and TTC39A alone or in combination are shown in Table 4 below and FIG. 5.

TABLE 4

| | | | Prediction | | |
|---|---|---|---|---|---|
| | Model | AUC | error | LOOCV.AUC | Accuracy |
| e0 | HER2 + PDL1 + RAD21_amp | 0.884 | 0.140 | 0.788 | 0.860 |
| e1 | ANKRD50_exprs | 0.739 | 0.386 | 0.704 | 0.614 |
| e2 | COX6C_exprs | 0.754 | 0.281 | 0.720 | 0.719 |
| e3 | DERL1_exprs | 0.739 | 0.263 | 0.708 | 0.737 |
| e4 | FLNB_exprs | 0.716 | 0.298 | 0.679 | 0.702 |
| e5 | GPRC5A_exprs | 0.690 | 0.368 | 0.646 | 0.632 |
| e6 | RAD21_exprs | 0.780 | 0.246 | 0.749 | 0.754 |
| e7 | RNF139_exprs | 0.774 | 0.281 | 0.737 | 0.719 |
| e8 | SAMD8_exprs | 0.720 | 0.351 | 0.693 | 0.649 |
| e9 | SERPINE1_exprs | 0.727 | 0.316 | 0.692 | 0.684 |
| e10 | SQLE_exprs | 0.737 | 0.246 | 0.713 | 0.754 |
| e11 | TTC39A_exprs | 0.702 | 0.316 | 0.653 | 0.684 |
| e12 | ANKRD50_exprs + RAD21_amp | 0.874 | 0.211 | 0.836 | 0.789 |
| e13 | COX6C_exprs + RAD21_amp | 0.837 | 0.228 | 0.779 | 0.772 |
| e14 | DERL1_exprs + RAD21_amp | 0.833 | 0.228 | 0.766 | 0.772 |
| e15 | FLNB_exprs + RAD21_amp | 0.800 | 0.193 | 0.757 | 0.807 |
| e16 | GPRC5A_exprs + RAD21_amp | 0.837 | 0.246 | 0.795 | 0.754 |
| e17 | RAD21_exprs + RAD21_amp | 0.799 | 0.211 | 0.690 | 0.789 |
| e18 | RNF139_exprs + RAD21_amp | 0.806 | 0.211 | 0.734 | 0.789 |
| e19 | SAMD8_exprs + RAD21_amp | 0.814 | 0.193 | 0.769 | 0.807 |
| e20 | SERPINE1_exprs + RAD21_amp | 0.860 | 0.246 | 0.820 | 0.754 |
| e21 | SQLE_exprs + RAD21_amp | 0.772 | 0.211 | 0.704 | 0.789 |
| e22 | TTC39A_exprs + RAD21_amp | 0.782 | 0.211 | 0.737 | 0.789 |
| e23 | COX6C_exprs + DERL1_exprs + | 0.858 | 0.193 | 0.811 | 0.807 |
| | SERPINE1_exprs | | | | |
| e24 | SQLE_exprs + COX6C_exprs + | 0.858 | 0.263 | 0.811 | 0.737 |
| | ANKRD50_exprs | | | | |
| e25 | ANKRD50_exprs + HER2 + | 0.939 | 0.123 | 0.911 | 0.877 |
| | PDL1 + RAD21_amp | | | | |
| e26 | COX6C_exprs + HER2 + | 0.909 | 0.140 | 0.810 | 0.860 |
| | PDL1 + RAD21_amp | | | | |
| e27 | DERL1_exprs + HER2 + | 0.913 | 0.158 | 0.865 | 0.842 |
| | PDL1 + RAD21_amp | | | | |
| e28 | FLNB_exprs + HER2 + | 0.905 | 0.140 | 0.868 | 0.860 |
| | PDL1 + RAD21_amp | | | | |
| e29 | GPRC5A_exprs + HER2 + | 0.913 | 0.140 | 0.844 | 0.860 |
| | PDL1 + RAD21_amp | | | | |
| e30 | RAD21_exprs + HER2 + | 0.884 | 0.140 | 0.788 | 0.860 |
| | PDL1 + RAD21_amp | | | | |
| e31 | RNF139_exprs + HER2 + | 0.891 | 0.140 | 0.788 | 0.860 |
| | PDL1 + RAD21_amp | | | | |
| e32 | SAMD8_exprs + HER2 + | 0.896 | 0.140 | 0.848 | 0.860 |
| | PDL1 + RAD21_amp | | | | |
| e33 | SERPINE1_exprs + HER2 + | 0.944 | 0.175 | 0.918 | 0.825 |
| | PDL1 + RAD21_amp | | | | |
| e34 | SQLE_exprs + HER2 + | 0.910 | 0.140 | 0.840 | 0.860 |
| | PDL1 + RAD21_amp | | | | |
| e35 | TTC39A_exprs + HER2 + | 0.906 | 0.140 | 0.866 | 0.860 |
| | PDL1 + RAD21_amp | | | | |
| e36 | COX6C_exprs + DERL1_exprs + | 0.898 | 0.211 | 0.840 | 0.789 |
| | SERPINE1_exprs + RAD21_amp | | | | |
| e37 | SQLE_exprs + COX6C_exprs + | 0.886 | 0.211 | 0.812 | 0.789 |
| | ANKRD50_exprs + RAD21_amp | | | | |
| e38 | TTC39A_exprs + SQLE_exprs + | 0.911 | 0.263 | 0.847 | 0.737 |
| | SERPINE1_exprs + DERL1_exprs + | | | | |
| | ANKRD50_exprs | | | | |
| e39 | COX6C_exprs + | 0.947 | 0.175 | 0.893 | 0.825 |
| | DERL1_exprs + | | | | |
| | SERPINE1_exprs + HER2 + | | | | |
| | PDL1 + RAD21_amp | | | | |
| e40 | SQLE_exprs + COX6C_exprs + | 0.935 | 0.140 | 0.880 | 0.860 |
| | ANKRD50_exprs + HER2 + | | | | |
| | PDL1 + RAD21_amp | | | | |
| e41 | TTC39A_exprs + SQLE_exprs + | 0.934 | 0.228 | 0.851 | 0.772 |
| | SERPINE1_exprs + DERL1_exprs + | | | | |
| | ANKRD50_exprs + RAD21_amp | | | | |
| e42 | SQLE_exprs + RNF139_exprs + | 0.865 | 0.316 | 0.750 | 0.684 |
| | RAD21_exprs + DERL1_exprs + | | | | |
| | COX6C_exprs + ANKRD50_exprs | | | | |
| e43 | TTC39A_exprs + SQLE_exprs + | 0.972 | 0.175 | 0.910 | 0.825 |
| | SERPINE1_exprs + DERL1_exprs + | | | | |
| | ANKRD50_exprs + HER2 + | | | | |
| | PDL1 + RAD21_amp | | | | |
| e44 | ATAD2_exprs + C8orf76_exprs + | 0.877 | 0.333 | 0.672 | 0.667 |
| | COX6C_exprs + DERL1_exprs + | | | | |
| | EIF3H_exprs + MRPL13_exprs + | | | | |
| | RAD21_exprs + RNF139_exprs + | | | | |
| | SERPINE1_exprs + SQLE_exprs + | | | | |
| | TRPS1_exprs | | | | |
| e45 | TTC39A_exprs + SQLE_exprs + | 0.933 | 0.246 | 0.741 | 0.754 |
| | SERPINE1_exprs + SAMD8_exprs + | | | | |
| | RNF139_exprs + RAD21_exprs + | | | | |
| | GPRC5A_exprs + FLNB_exprs + | | | | |
| | DERL1_exprs + COX6C_exprs + | | | | |
| | ANKRD50_exprs | | | | |

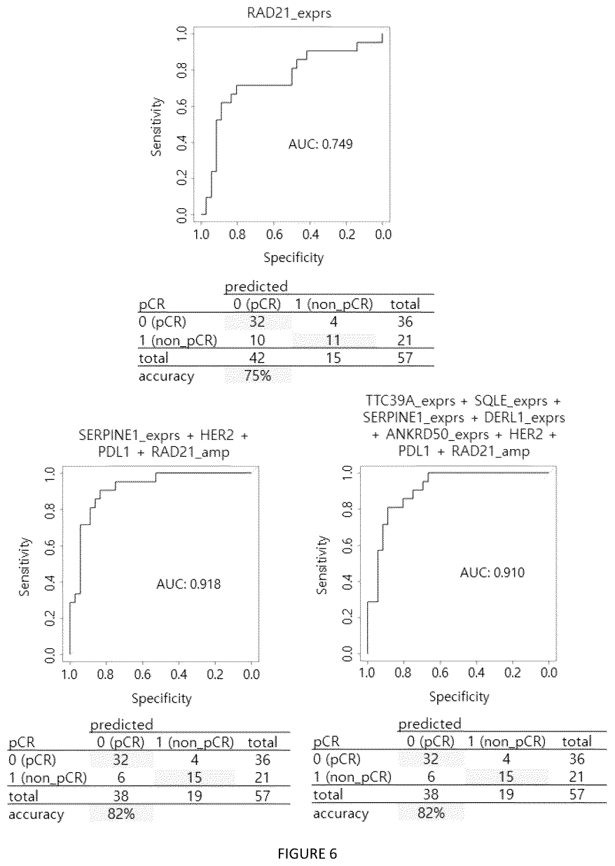

As a result, the e43 model (TTC39A_exprs+SQLE_exprs+SERPINE1_exprs+DERL1_exprs+ANKRD50_exprs+HER2+PDL1+RAD21_amp) including all of the DNA marker, protein markers and RNA markers exhibited the highest AUC, and the e33 model (SERPINE1_exprs+HER2+PDL1+RAD21_amp) including only SERPINE1_exprs among the RNA markers exhibited the best performance based on LOOCV.AUC (see FIG. 6).

These results suggest that, when the RAD21 gene, in particular, the presence or absence of DNA mutation in the RAD21 gene, is used together with the DNA markers and the RNA markers, it is possible to more accurately predict prognosis of drug treatment of HER2 early-positive breast cancer patients to whom neoadjuvant chemotherapy is applicable.

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

What is claimed is:

1. A composition for predicting prognosis of treatment of HER2-positive breast cancer, the composition containing an agent for detecting RAD21 gene.

2. The composition of claim 1, wherein the treatment of HER2-positive breast cancer is treatment of HER2-positive early breast cancer patients to whom neoadjuvant chemotherapy is applicable.

3. The composition of claim 2, wherein the neoadjuvant chemotherapy comprises administering: docetaxel, paclitaxel, or nanoparticle albumin-bound (nab) paclitaxel; trastuzumab; pertuzumab; and atezolizumab, nivolumab or pembrolizumab.

4. The composition of claim 1, wherein the agent is one for detecting a DNA mutation in the RAD21 gene, a RNA expression level of the RAD21 gene, or a combination thereof.

5. The composition of claim 4, wherein the DNA mutation in the RAD21 gene is at least one selected from the group consisting of: a single nucleotide mutation; deletion, substitution, insertion, or combination thereof, of 1 to 50 nucleotides; and copy number variation.

6. The composition of claim 1, wherein the agent is at least one selected from the group consisting of primers, probes, and antisense nucleotides, which bind to DNA or RNA of the RAD21 gene.

7. The composition of claim 1, further containing an agent for measuring an expression level of HER2 gene or protein.

8. The composition of claim 1, further containing an agent for measuring an expression level of PDL1 gene or protein.

9. The composition of claim 7, wherein the agent is at least one selected from the group consisting of primers, probes, antisense nucleotides, antibodies, oligopeptides, ligands, PNAs, and aptamers, which bind to the HER2 or PDL1 gene or protein.

10. The composition of claim 1, further containing an agent for measuring an RNA expression level of at least one gene selected from the group consisting of ANKRD50, COX6C, DERL1, FLNB, GPRC5A, RNF139, SAMD8, SERPINE1, SQLE, and TTC39A genes.

11. The composition of claim 10, wherein the agent is a primer or probe that binds to the RNA sequence.

12. A kit for predicting prognosis of treatment of HER2-positive breast cancer, the kit comprising the composition of claim 1.

13. A method of providing information for predicting prognosis of treatment of HER2-positive breast cancer, the method comprising steps of:
a) detecting RAD21 gene in a biological sample isolated from a subject; and
b) comparing a DNA mutation level, RNA expression level, or combination thereof, of the detected RAD21 gene with that in a control group.

14. The method of claim 13, wherein the subject in step a) is a HER2-positive early breast cancer patient to whom neoadjuvant chemotherapy is applicable.

15. The method of claim 14, wherein the neoadjuvant chemotherapy comprises administering: docetaxel, paclitaxel or nanoparticle albumin-bound (nab) paclitaxel; trastuzumab; pertuzumab; and atezolizumab, nivolumab or pembrolizumab.

16. The method of claim 13, wherein the biological sample in step a) is at least one selected from the group consisting of blood, plasma, serum, lymphatic fluid, saliva, urine, and tissue.

17. The method of claim 13, further comprising, after step a) or b), steps of:
a-1) of measuring an expression level of HER2 gene or protein in the biological sample; and
b-1) comparing the measured expression level of the HER2 gene or protein with that in the control group.

18. The method of claim 13, further comprising, after step a) or b), steps of:
a-2) of measuring an expression level of PDL1 gene or protein in the biological sample; and
b-2) comparing the measured expression level of the PDL1 gene or protein with that in the control group.

19. The method of claim 13, further comprising, after step a) or b), steps of:
a-3) measuring an RNA expression level of at least one gene selected from the group consisting of ANKRD50, COX6C, DERL1, FLNB, GPRC5A, RNF139, SAMD8, SERPINE1, SQLE, and TTC39A genes, in the biological sample; and
b-3) comparing the measured RNA expression level of the gene with that in the control group.

20. The method of claim 13, wherein the DNA mutation level, gene or RNA expression level, or protein expression level of the gene is measured by at least one method selected from the group consisting of fluorescence in situ hybridization, chromatin immunoprecipitation, next-generation sequencing, polymerase chain reaction (PCR), reverse transcription polymerase chain reaction (RT-PCR), competitive RT-PCR, real-time PCR, real-time RT-PCR, nuclease protection assay, in situ hybridization, DNA or RNA microarray, Northern blotting, enzyme-linked immunosorbent assay, Western blotting, radioimmunoassay, radioimmunodiffusion, immunoprecipitation, flow cytometry, immunohistochemistry, immunofluorescence, and protein microarray.

21. The method of claim 13, wherein, in the step of comparing with that of the control group, it is determined that, when one or more of the following i) and ii) and one or more of the following iii) to v) are satisfied in the subject compared to the control group, the prognosis of treatment of the HER2 positive breast cancer patient is good:
i) a low DNA mutation level of the RAD21 gene;
ii) a low RNA expression level of RAD21 gene;
iii) a high expression level of the HER2 gene or protein;
iv) a high expression level of the PDL1 gene or protein; and
v) a low RNA expression level of at least one gene selected from the group consisting of ANKRD50, COX6C, DERL1, FLNB, GPRC5A, RNF139, SAMD8, SERPINE1, SQLE, and TTC39A genes.
